(19) United States
(12) Patent Application Publication
(10) Pub. No.: US 2011/0269782 A1
(43) Pub. Date: Nov. 3, 2011

(54) USE OF NMDA ACTIVITY ENHANCERS TO TREAT HYPERSOMNIA, REDUCED WAKEFULNESS OR REDUCED VIGILANCE

(76) Inventors: Magnus Ivarsson, Hertfordshire (GB); Nick Brandon, Hertfordshire (GB); Peter H. Hutson, Hertfordshire (GB)

(21) Appl. No.: 13/120,275

(22) PCT Filed: Sep. 23, 2009
(86) PCT No.: PCT/GB09/51235

§ 371 Date: Jul. 19, 2011

(30) Foreign Application Priority Data
Sep. 23, 2008 (GB) 0817379.1

(51) Int. Cl. A61K 31/522; A61P 25/26; A61P 25/00; C07D 495/04; A61K 31/407

(52) U.S. Cl. 514/263.32; 548/453; 514/412

(57) ABSTRACT

The present invention discloses the use of an enhancer of NMDA activity that acts via the glycine modulatory site to treat hypersomnia, reduced wakefulness or reduced vigilance as well as methods for preparing such compounds.

The present invention relates to the use of an enhancer of NMDA activity that acts via the glycine modulatory site to treat hypersomnia, reduced wakefulness or reduced vigilance.

Depoortère et al., Neuropsychopharmacol., 2005, 1-23 relates to glycine transporter-1 (GlyT1), SSR504734, a selective and reversible inhibitor of human, rat and mouse GlyT1. It discloses that at 30 mg/kg SSR504734 induced a long-lasting reduction in paradoxical sleep (PS) (characterised by hypersynchronization of the theta rhythm of the visual cortex). This was mainly the consequence of an increased latency time to onset of the first episode and, to a lesser extent, of the number of episodes of PS. The duration of wakefulness was also increased but this effect was only for the first two hours. 24 h after drug challenge sleep parameters had returned to control levels.

Despite the above comments, no conclusion is drawn that the reduction in PS is due to the GlyT1 inhibitor enhancing wakefulness. Certainly, no conclusions are made indicating that an enhancement of wakefulness can be produced by any compound enhancing NMDA activity by acting through the glycine modulatory site.

It has now surprisingly been found that a wide range of compounds can enhance wakefulness such as GlyT1 inhibitor, D-serine and analogues thereof, and DAO (D-amino acid oxidase) inhibitors.

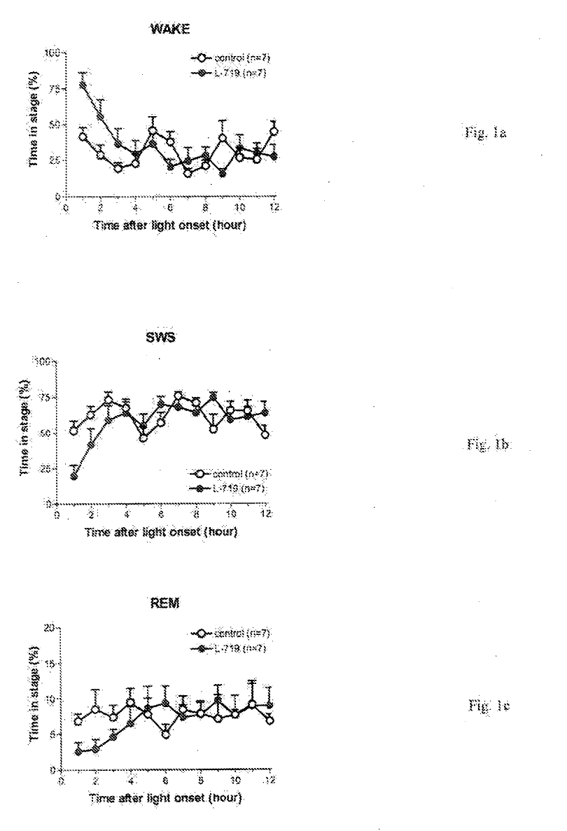

FIG. 1 shows the effects on wakefulness, slow-wave sleep and REM sleep of 3 mg/kg (p.o.) 2,4-dichloro-N-(4-cyclopropylmethane-sulfonyl-1-cyclopropylmethylcyclohexylmethyl)benzamide (Merck 1) administered to rats. It can be seen that there was a significant decrease in REM sleep (54±7%; mean±SEM) and a trend towards increasing the time spent in WAKE (93±43%; p=0.06).

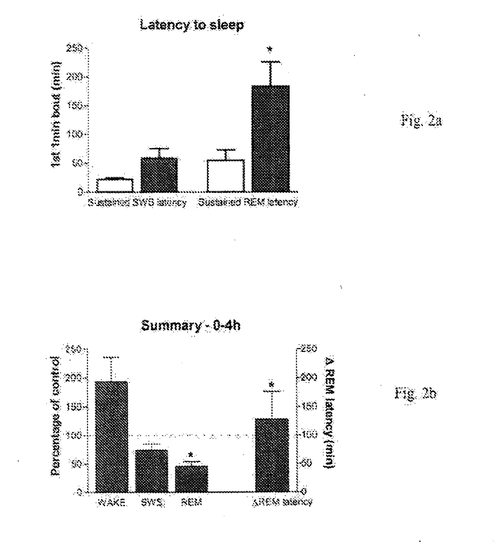

FIG. 2 shows the latency to sleep for Merck 1 administered as above. There was a significant increase in REM latency. In addition to these findings significant decreases in the number of entries into SWS (26±8%) and REM (45±9%) were recorded.

In the above experiment rats implanted with a two channel telemetry transmitter to record EEG and EMG simultaneously were dosed with either Merck 1 (nanosuspension 3 mg/kg; 5 ml/kg; p.o.) or vehicle (1.4% W/V hydroxypropylmethylcellulose 0.2% W/V sodium docusate; p.o.) at light on-set in a cross-over design. EEG and EMG recordings were collected for at least 12 hours. Data was converted with LoadDSI.s2s and BatchEEGpower2.s2s scripts (CED) and every 12 s epoch was automatically classified as either WAKE, SWS or REM (PS) sleep with Modulus analysis of sleep v7.xlt. Statistical analysis was done with Student's t-test for paired data. REM latency was defined as the first>1 min REM bout.

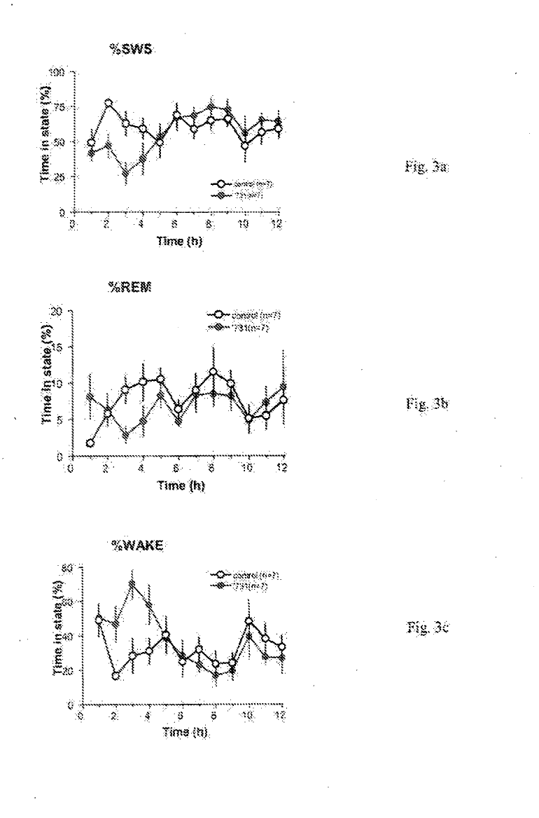

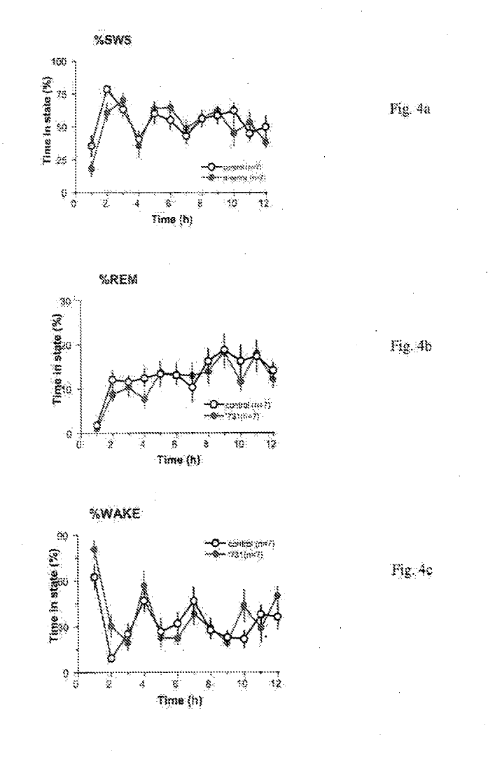

Similar data is shown for D-amino acid oxidase inhibitor 4H-furo[3,2-b]pyrrole-5-carboxylic acid (Merck 2) in FIG. 3 and D-serine in FIG. 4 at 100 mg/kg. In this experiment rats implanted with a two channel telemetry transmitter to record EEG and EMG simultaneously were dosed with either drug (30, 100, 300 mg/kg d-serine or 25 mg/kg Merck 2) or vehicle (water or 0.5% MC) intraperitoneally at light on-set in a cross-over design. EEG and EMG recordings were collected for at least 12 hours. Data was converted with LoadDSI.s2s and BatchEEGpower2.s2 s scripts (CED) and every 12 s epoch was automatically classified as either WAKE, SWS or REM (PS) sleep with Modulus analysis of sleep v5 REM incl entries.XLT. Preliminary statistical analysis was done with Student's t-test for paired data. Significance level p<0.05.

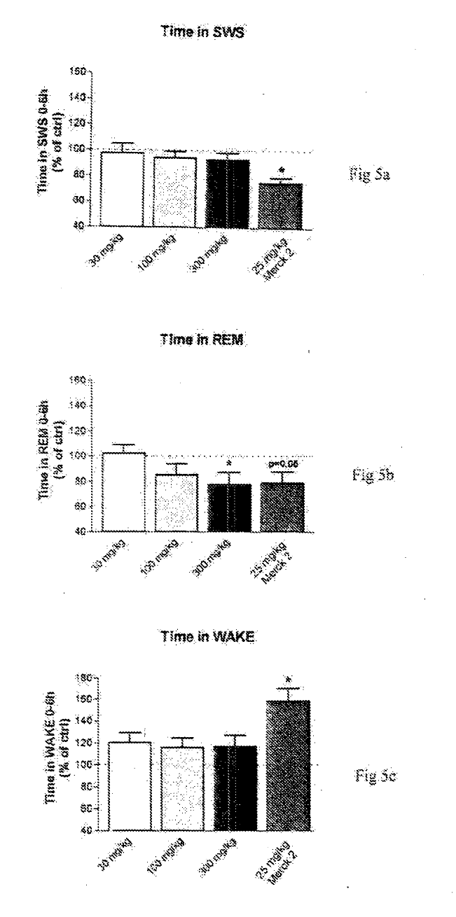

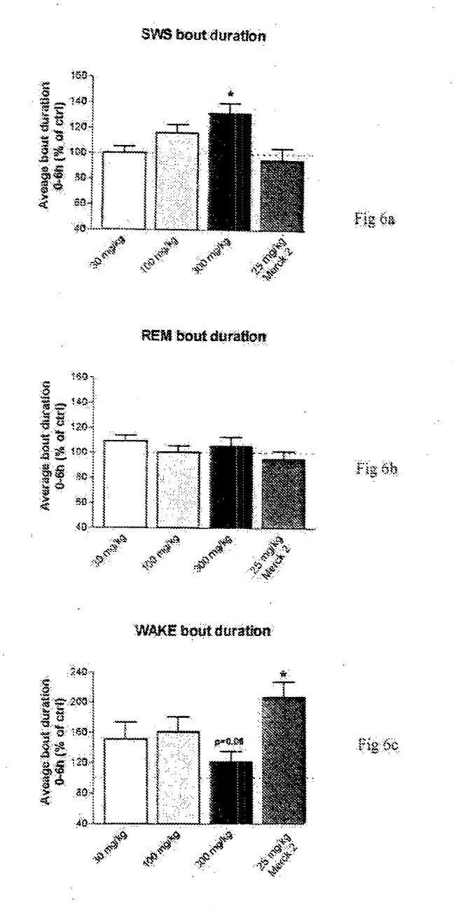

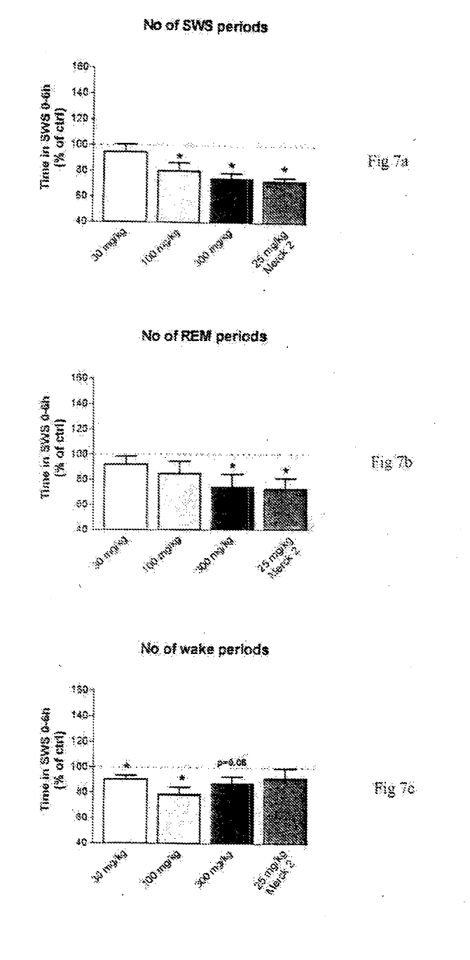

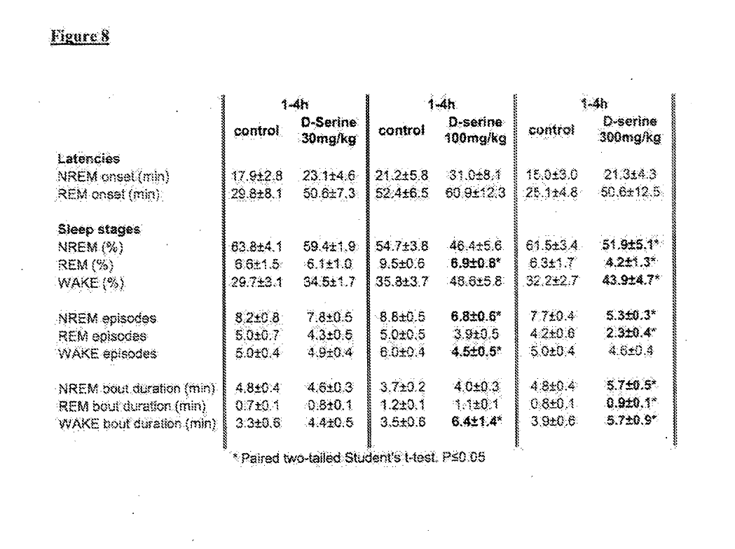

The effects of D-serine at 30, 100 and 300 mg/kg i.p. and of Merck 2 at 25 mg/kg i.p. are shown in FIGS. 5, 6 and 7. FIG. 5 shows the time in vigilance stage, FIG. 6 the bout duration and FIG. 7 the number of bouts. The underlying data is shown in FIG. 8.

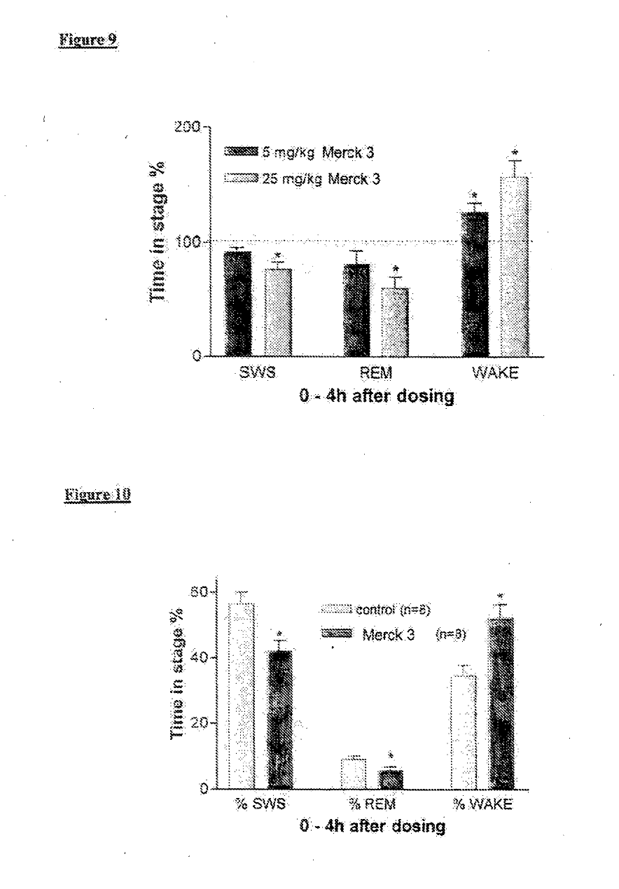

FIGS. 9 and 10 show the effect of DAO inhibitor 4H-thieno[3,2-b]pyrrole-5-carboxylic acid (Merck 3) on rat sleep parameters as a percentage of control over the first 4 h for doses of 5 mg/kg i.p. and 25 mg/kg i.p. respectively.

Thus the present invention provides a compound which enhances NMDA activity via the glycine modulatory site for treating hypersomnia, reduced wakefulness or reduced vigilance.

Also provided is a compound that positively modulates levels of d-serine, glycine or d-alamine for treating hypersomnia, reduced wakefulness or reduced vigilance.

Accordingly the present invention provides the use of a compound which enhances NMDA activity via the glycine modulatory site for the manufacture of a medicament for treating hypersomnia, reduced wakefulness or reduced vigilance.

In another embodiment there is provided the use of a compound that positively modulates levels of d-serine, glycine or d-alamine for the manufacture of a medicament for treating hypersomnia, reduced wakefulness or reduced vigilance.

In particular the compound is a GlyT1 inhibitor, a DAO inhibitor or D-serine or an analogue thereof.

The present invention also provides a method of treating a subject suffering from hypersomnia, reduced wakefulness or reduced vigilance by administering to that subject a therapeutically effective amount of a compound which enhances NMDA activity via the glycine modulatory site.

There is thus also provided a method of treating a subject suffering from reduced wakefulness, hypersomnia or reduced vigilance by administering to that subject a compound that positively modulates levels of d-serine, glycine or d-alamine.

The present invention also provides combinations of the above compounds with other wake promoting drugs such as caffeine or modafinil.

Without wishing to be bound by theory, it is believed that the present invention works by increasing the amount of substrates, such as D-amino acids and glycine, that bind to the glycine site thereby enhancing the amount of NMDA and promoting wakefulness.

One embodiment of the present invention concerns treating hypersomnia. Another embodiment concerns treating reduced wakefulness. A third embodiment concerns treating reduced vigilance.

Hypersomnia is synonymous with increased daytime sleepiness. The effect of the compounds used in the present invention against hypersomnia can be tested in animals in any convenient model of increased sleepiness. For example, the regular sleep period of an animal can be interrupted and then their increased sleepiness the following day can be studied.

GlyT1 inhibitors of use in the present invention are compounds active in the following assay. Human placental choriocarcinoma cells (JAR cells (ATCC No. HTB-144)) endogenously expressing GlyT1 were cultured in 96-well Cytostar scintillating microplates (Amersham Biosciences) in RPMI 1640 medium containing 10% fetal calf serum in the presence of penicillin (100 micrograms/milliliter) and streptomycin (100 micrograms/milliliter). Cells were grown at 37° C. in a humidified atmosphere of 5% CO2 for 40-48 hours before the assay. Culture medium was removed from the Cytostar plate, and JAR cells were incubated with 30 microliters of TB1A buffer (120 mM NaCl, 2 mM KCl, 1 mM CaCl₂, 1 mM MgCl₂, 10 mM HEPES, 5 mM L-alanine, pH 7.5 adjusted with Tris base) with or without the compounds of the present invention for 1 minute. Then 30 microliters of [¹⁴C]-glycine diluted with TB1A was added to each well to give a final concentration of 10 micromolar. After incubation at room temperature for 3 hours, the Cytostar scintillating microplates were sealed and counted on a Top Count scintillation counter (Packard). Non-specific uptake of [¹⁴C]-glycine was determined in the presence of 10 mM unlabeled glycine. [¹⁴C]taurine uptake experiments were performed according to the same protocol except that 10 mM unlabeled taurine was used to determine non-specific uptake. To determine potencies, a range of concentrations of the compounds used in present invention was added to the cells, followed by the fixed concentration of [¹⁴C]glycine. The concentration of the compound that inhibited half of the specific uptake of [¹⁴C]glycine (IC₅₀ value) was determined from the assay data by non-linear curve fitting.

In particular, the compounds inhibit specific uptake of [¹⁴C]glycine in the aforementioned assay, generally with an IC₅₀ value of less than about 10 micromolar. Preferred compounds have activity in inhibiting specific uptake of [¹⁴C]glycine in the aforementioned assay with an IC₅₀ value of less than about 1 micromolar. These compounds were selective for [¹⁴C]glycine uptake (by GlyT1 in the JAR cells) compared to [¹⁴C]taurine uptake (by the taurine transporter TauT in the JAR cells). Such a result is indicative of the intrinsic activity of the compounds in use as inhibitors of GlyT1 transporter activity.

DAO inhibitors of use in the present invention can be identified by the following assay: CHO cells stably expressing human D-amino acid oxidase were grown in F12/Ham glutamax medium containing 10% FBS, 1×pen/strep and 1 mg/ml G418. On the day of assay, cells were washed with PBS, harvested and spun at 1000 rpm for 5 mins before resuspending in assay buffer (HBSS containing 1M CaCl₂, 1M MgCl₂ and 1M Hepes, pH 7.4) at 8.6×10⁵/ml. 35 ul cell suspension was added to 5 ul test compound in a 384 well plate. The assay was initiated by the addition of 10 ul assay buffer containing 2.5% amplex red (Molecular Probes), 6% horse radish peroxidase and 25% 1M D-serine. Plates were incubated for 2 hours at 37° C. and fluorescence (excitation 544 nm, emission 590 nm) read using a Cytofluor plate reader. Compounds should have activity at below the one micromolar level.

The compounds used in the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art.

An appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Examples of GlyT1 inhibitors can be found in WO05/046601, WO05/094514, WO05/107469, WO05/110983, WO06/039221, WO06/067529, WO06/131711, WO06/131713, WO06/134341, WO07/041025, WO07/053400 and WO07/060484, all in the name of Merck Sharp & Dohme Ltd and/or Merck & Co., Inc.

Exemplary GlyT1 inhibitors include, from WO06/131713, 2,4-dichloro-N-(4-cyclopropylmethanesulfonyl-1-cyclopropylmethylcyclohexyl-methyl)benzamide; N-({1-(cyclopropylmethyl)-4-[(cyclopropylmethyl)sulfonyl]cyclohexyl}methyl)-2,4-bis(trifluoromethyl)pyrimidine-5-carboxamide; N-({1-(cyclopropylmethyl)-4-[(cyclopropylmethyl)sulfonyl]cyclohexyl}methyl)-4-methyl-2-(trifluoromethyl)pyrimidine-5-carboxamide; 2-chloro-N-({1-(cycloproplylmethyl)-4-[(cyclopropylmethyl)sulfonyl]cyclohexyl}methyl)-4-fluorobenzamide; 2,6-dichloro-N-({1-(cyclopropylmethyl)-4-[(cyclopropylmethyl)sulfonyl]cyclohexyl}methyl)nicotinamide; 2,4,6-trichloro-N-({1-(cyclopropylmethyl)-4-[(cyclopropylmethyl)sulfonyl]cyclohexyl}methyl)benzamide; 2-chloro-N-({1-(cyclopropylmethyl)-4-[(cyclopropylmethyl)sulfonyl]cyclohexyl}methyl)-4-(trifluoromethyl)benzamide; N-({1-(cyclopropylmethyl)-4-[(cyclopropylmethyl)sulfonyl]cyclohexyl}methyl)-4-(methylsulfonyl)benzamide; and from WO06/039221, 2-amino-6-chloro-N-{[4-(cyclopropylmethyl)-1-(propylsulfonyl)piperidin-4-yl]methyl}benzamide; 2-chloro-N-{[4-(cyclopropylmethyl)-1-(propylsulfonyl)piperidin-4-yl]methyl}-6-fluorobenzamide; 2-chloro-N-{[4-(cyclopropylmethyl)-1-(ethylsulfonyl)piperidin-4-yl]methyl}-6-fluorobenzamide; 2-chloro-N-({4-(cyclopropylmethyl)-1-[(2,2,2-trifluoroethyl)sulfonyl]piperidin-4-yl}methyl)-4-fluorobenzamide; N-{[4-(cyclopropylmethyl)-1-(ethylsulfonyl)piperidin-4-yl]methyl}cyclohexanecarboxamide; N-{[4-(cyclopropylmethyl)-1-(ethylsulfonyl)piperidin-4-yl]methyl}-2-(trifluoromethyl)benzamide; N-{[4-(cyclopropylmethyl)-1-(ethylsulfonyl)piperidin-4-yl]methyl}-2,4-difluorobenzamide; N-{[4-(cyclopropylmethyl)-1-(ethylsulfonyl)piperidin-4-yl]methyl}-2,6-difluorobenzamide; 2-chloro-N-{[4-(cyclopropylmethyl)-1-(ethylsulfonyl)piperidin-4-yl]methyl}-4,6-difluorobenzamide; N-{[4-(cyclopropylmethyl)-1-(ethylsulfonyl)piperidin-4-yl]methyl}-2,3-difluorobenzamide; 2,4-dichloro-N-{[4-(cyclopropylmethyl)-1-(propylsulfonyl)piperidin-4-yl]methyl}benzamide; N-{[4-(cyclopropylmethyl)-1-(propylsulfonyl)piperidin-4-yl]methyl}-2-furamide; 2-chloro-N-{[4-(cyclopropylmethyl)-1-(isopropylsulfonyl)piperidin-4-yl]methyl}-4-fluorobenzamide; 2,4-dichloro-N-{[4-(cyclopropylmethyl)-1-(cyclopropylsulfonyl)piperidin-4-yl]methyl}benzamide; and their pharmaceutically acceptable salts.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of use in the present invention are meant to also include the pharmaceutically acceptable salts.

Examples of DAO inhibitors include 4H-thieno[3,2-b]pyrrole-5-carboxylic acid and 4H-furo[3,2-b]pyrrole-5-carboxylic acid, (see WO07/039773 in the names of Merck Sharp & Dohme Ltd and Merck & Co., Inc.), 1H-indole-2-carboxylic acid (see WO03/039540 in the name of Sepracor Inc.) and 4-[2-(4-chlorophenyl)ethyl]-1H-pyrrole-2-carboxylic acid (see US2005143443 in the name of Sepracor Inc.).

In the present invention ‘paradoxical sleep’, ‘wakefulness’ and ‘slow wake sleep’ have the same meanings as REM, WAKE and NREM respectively in Ivarsson et al, Eur. J. Pharmacol., 522 (2005) 63-71. That is, these terms include both brain activity (EEG) changes and muscle activity (EMG). Representative examples of these sleep patterns can be seen in FIG. 1A of Ivarsson et al. The different vigilance stages are shown in FIG. 1B. Paradoxical sleep shows desynchronised low amplitude EEG activity with low or absent EMG activity. Wakefulness is characterised by desynchronised, low amplitude EEG activity with increased EMG activity. Slow wave sleep displays synchronized, high amplitude EEG activity accompanied by low muscle activity.

The following examples illustrate the present invention.

### EXAMPLE 1

Effect of DAO-Inhibitor Merck 3 on Rat Sleep Parameters at 5 mg/kg i.p.

Merck 3 is a novel DAO-inhibitor. The aim of this study was to assess the effect of the acute dose of 5 mg/kg Merck 3 i.p. on rat sleep parameters and see how it compared to the higher dose of 25 mg/kg i.p.

Rats implanted with a two channel telemetry transmitter to record EEG and EMG simultaneously were dosed acutely with either Merck 3 (5 mg/kg; i.p.) or vehicle (distilled water; i.p.) at light onset in a cross-over design. EEB and EMG recordings were collected for at least 12 hours. Data was converted with LoadDSI.s2s and BatchEEGpower2.s2s scripts (CED) and every 12 s epoch was automatically classified as either WAKE, SWS or REM (PS) sleep and Modulus analysis of sleep v7 REM incl entries and tot.xlt. Preliminary statistical analysis was done with Student's t-test for paired data. Significance level p<0.05.

Merck 3 (5 mg/kg; i.p.) significantly reduced the bout duration of SWS in the 7 to 12 h time period. The dose also significantly increased the time spent awake in the first four hours after dosing. These effects are similar to what was observed at 25 mg/kg i.p. but so to a smaller scale. Unlike 25 mg/kg i.p. 5 mg/kg i.p. significantly increased the latency to REM sleep. The results are shown in FIG. 9.

### EXAMPLE 2

### Effect of DAO-Inhibitor Merck 3 on Rat Sleep Parameters

The aim of this study was to assess the effect of the acute dose of 25 mg/kg Merck 3 i.p. on rat sleep parameters.

Rats implanted with a two channel telemetry transmitter to record EEG and EMG simultaneously were dosed acutely with either Merck 3 (25 mg/kg; i.p.) or vehicle (distilled water; i.p.) at light onset in a cross-over design. EEG and EMG recordings were collected for at least 12 hours. Data was converted with LoadDSI.s2s and BatchEEGpower2.s2s scripts (CED) and every 12 s epoch was automatically classified as either WAKE, SWS or REM (PS) sleep with Modulus analysis of sleep v6 REM incl entries and tot.xlt. Preliminary statistical analysis was done with Student's t-test for paired data. Significance level p<0.05.

Merck 3 (25 mg/kg; i.p.) produced all its significant effects on sleep architecture in the first four hours, with a decrease in both time spent in SWS and REM. Correspondingly, Merck 3 treated animals were significantly more awake (in the first 4 h) compared to the control. The amount of time spent in each sleep state and the latency to SWS and REM were unaltered.

1-9. (canceled)

10. A compound which enhances NMDA activity via the glycine modulatory site or that positively modulates levels of d-serine, glycine or d-alanine.

11. The compound of claim 10 that is a GlyT1 inhibitor, a DAO inhibitor or D-serine or an analogue thereof.

12. A combination comprising the compound of claim 10 and another wake promoting drug.

13. The combination of claim 12, wherein the wake promoting drug is caffeine or modafinil.

14. A method of treating a subject suffering from hypersomnia, reduced wakefulness or reduced vigilance by administering to the subject a therapeutically effective amount of a compound which enhances NMDA activity via the glycine modulatory site or that positively modulates levels of d-serine, glycine or d-alamine.

15. The method of claim 14, wherein the compound is a GlyT1 inhibitor, a DAO inhibitor or D-serine or an analogue thereof.

16. The method of claim 14, wherein the compound is administered in combination with another wake promoting drug.

17. The method of claim 16, wherein the wake promoting drug is caffeine or modafinil.

18. The method of claim 16, wherein the administration is simultaneous, separate or sequential administration.
